SEQUENCE LISTING (23 sequences)

SEQ ID NO: 1 - protein, 14 aa, Artificial
  SV40 large T-antigen core NLS (PKKKRKV) with arbitrarily chosen flanking amino acids (glycine and serine)
GSGSPKKKRKVGSG

SEQ ID NO: 2 - protein, 14 aa, Artificial
  SV40 large T-antigen core NLS (PPKKKRKV) with EED N-terminal flanking region of the NLS form Polyoma virus VP2-protein
EEDTPPKKKRKVED

SEQ ID NO: 3 - protein, 22 aa, Artificial
  NLS of the Influenza virus nuclear protein
MASQGTKRSYEQMETDGERQYC

SEQ ID NO: 4 - protein, 21 aa, Artificial
  Mutated part of the SV40 large T-antigen bearing the core NLS
GKPTADDQHSTPPKKKRKVED

SEQ ID NO: 5 - protein, 27 aa, Artificial
  Natural part of the SV40 large T-antigen bearing  the core NLS
GKPSSDDEATADSQHSTPPKKKRKVED

SEQ ID NO: 6 - protein, 14 aa, Artificial
  4-fold positively charged NLS1
ESGSPKKKRKVGSG

SEQ ID NO: 7 - protein, 14 aa, Artificial
  4-fold positively charged NLS1
GSGSPKKKRKVDSG

SEQ ID NO: 8 - protein, 14 aa, Artificial
  3-fold positively charged NLS1
ESGSPKKKRKVDSG

SEQ ID NO: 9 - protein, 14 aa, Artificial
  3-fold positively charged NLS1
EDGSPKKKRKVGSG

SEQ ID NO: 10 - protein, 14 aa, Artificial
  3-fold positively charged NLS1
GSGEPKKKRKVDSG

SEQ ID NO: 11 - protein, 14 aa, Artificial
  3-fold positively charged NLS2
GSDTPPKKKRKVES

SEQ ID NO: 12 - protein, 14 aa, Artificial
  3-fold positively charged NLS2
GSGTPPKKKRKVED

SEQ ID NO: 13 - protein, 14 aa, Artificial
  4-fold positively charged NLS2
GESTPPKKKRKVGS

SEQ ID NO: 14 - protein, 14 aa, Artificial
  4-fold positively charged NLS2
GSGTPPKKKRKVES

SEQ ID NO: 15 - protein, 21 aa, Artificial
  3-fold positively charged SV21
GKPTAGDQHSTPPKKKRKVED

SEQ ID NO: 16 - protein, 21 aa, Artificial
  4-fold positively charged SV21
GKPTADDQHSTPPKKKRKVSG

SEQ ID NO: 17 - protein, 20 aa, Artificial
  DNA-anchoring peptide
KKKKKKKKKKKKKKKKGGGS

SEQ ID NO: 18 - protein, 16 aa, Artificial
  DNA-anchoring peptide without spacer U3
KKKKKKKKKKKKKKKK

SEQ ID NO: 19 - protein, 24 aa, Artificial
  DNA-anchoring peptide
KKKKKKKKKKKKKKKKKKKKGGGS

SEQ ID NO: 20 - protein, 29 aa, Artificial
  DNA-anchoring peptide
KKKKKKKKKKKKKKKKKKKKKKKKKGGGS

SEQ ID NO: 21 - protein, 22 aa, Artificial
  DNA-anchoring peptide
RKGKKVRKGKKVRKGKKVGGGS

SEQ ID NO: 22 - protein, 28 aa, Artificial
  DNA-anchoring peptide
RKGKKVRKGKKVRKGKKVRKGKKVGGGS

SEQ ID NO: 23 - protein, 24 aa, Artificial
  DNA-anchoring peptide
RKKRKRKKRKRKKRKRKKRKGGGS

(19) United States
(12) Patent Application Publication
(10) Pub. No.: US 2009/0253206 A1
(43) Pub. Date: Oct. 8, 2009

(54) NUCLEAR TRANSPORT AGENT AND METHOD FOR PRODUCING SAID AGENT

(75) Inventors: Gregor Siebenkotten, Frechen (DE); Bodo Ortmann, Cologne (DE)

Correspondence Address: JOYCE VON NATZMER;PEQUIGNOT + MYERS LLC, 200 Madison Avenue, Suite 1901, New York, NY, 10016, US

(73) Assignee: LONZA COLOGNE AG, Cologne (DE)

(21) Appl. No.: 12/440,416

(22) PCT Filed: Sep. 13, 2007
(86) PCT No.: PCT/EP2007/007988

§ 371 Date: Mar. 6, 2009

(30) Foreign Application Priority Data
Sep. 13, 2006 (EP) 06019155.8

(51) Int. Cl. C12N 15/87; C07K 2/00; C07C 57/00; C07K 7/00; C07K 14/00

(52) U.S. Cl. 435/455; 530/300; 554/1; 530/326; 530/325; 530/324; 530/327

(57) ABSTRACT

The present invention relates to a transport agent for transporting nucleic acids into eukaryotic cells and a method for producing said agent. The invention further concerns methods for transporting nucleic acids into eukaryotic cells using the transport agent according to the invention. The present invention provides an alternative transport agent and a method which are effective to allow an efficient transport of nucleic acids into eukaryotic cells. The transport agent comprises a complex forming moiety that is capable of forming complexes with at least one nucleic acid molecule and condensing said nucleic acid molecule, and at least one nuclear localization moiety comprising at least one nuclear localization signal and having an approximately neutral net charge.

### FIELD OF THE INVENTION

The present invention relates to a transport agent for transporting nucleic acids into eukaryotic cells and a method for producing said agent. The invention further concerns methods for transporting nucleic acids into eukaryotic cells using the transport agent according to the invention.

### BACKGROUND OF THE INVENTION

Genetic material is active in the nucleus. The transport there can occur coincidentally during cell division when the nuclear envelope temporarily disintegrates in the course of mitosis or it has to be carried out actively. Thus, the active transport into the nucleus of living cells is necessary for the transfer of genetic material into all of those cells that do not divide in the period before the intended expression of the genetic material. A nuclear transport system for nucleic acids is very important because it is effective to allow the efficient transfer of DNA into those cells that divide rarely or not at all. Most primary cells belong to this group. Primary cells are of the highest scientific interest for two reasons. First, these cells that have freshly been isolated from the organism much better reflect the functional state of the cell type than cell lines derived from them. Second, they are the target cells for gene therapy. In addition, a nuclear transport system increases the efficiency of DNA transfer into established cell lines by enabling those cells to express transferred genetic material that have not divided in the period of time between start of transfer and analysis.

The bilayer membrane that envelops the nucleus includes pores. Small molecules can traverse these pores by diffusion. In order to be able to enter the nucleus, proteins larger than about 50 kDa need a nuclear localization signal (NLS) that has to be recognized by the transport machinery. An NLS is a signal peptide that mediates transfer of a cargo, which may be another peptide, from the cytoplasm into the nucleus of a cell. Typically, a functional signal consists of four to eight amino acids, is rich in positive amino acids Arginine and Lysine and contains Prolines. It is strongly conserved in evolution so that mammalian NLS also function in yeast. A classical NLS is, for example, the SV40 large T-antigen core sequence PKKKRKV. Heterologous NLS may also be used as a tool to transport target molecules into the nucleus. For this purpose, NLS can be incorporated into the sequences of cytoplasmic proteins at relatively random positions or NLS peptides can be coupled chemically to proteins or even gold particles (reviewed in Görlich, 1998). An overview of NLS is given by T. Boulikas (1993).

Nagasaki et al. (2003) demonstrate that nuclear localization of plasmid DNA cannot be achieved by injecting conjugates of DNA and classical cationic NLS into the cytoplasm of cells. On the other hand, several artificial systems have been described that are supposed to increase transfection efficiency with the help of peptides or proteins containing nuclear localization signals.

Subramanian et al. (1999) disclose a conjugate of a non-classical NLS and a cationic peptide scaffold derived from a scrambled sequence of the SV40 T-antigen consensus NLS in order to improve the final step of nuclear import with lipofection of non-dividing cells.

U.S. Pat. No. 5,670,347 discloses a peptide that consists of a DNA-binding basic region, a flexible hinge region and an NLS. As DNA binding is achieved by the amino acids' positive charges also in this case, the reagent forms complexes with the DNA that are meant to serve for the transport across the cellular membrane at the same time. It is not evident why the NLS sequence should not participate in the binding of DNA, so that the actual signal for the nuclear transport proteins is likely to be masked by the DNA as long as the peptide is linked to it. Moreover, the complexes generated may become very large, cf. amongst others Emi et al. (1997), what would impair transport through the nuclear pores, cf. amongst others Yoneda et al. (1987, 1992).

WO 01/38547 A2 discloses polypeptides for transfer of molecules into eukaryotic cells that comprise at least two peptide monomers which each include a nuclear localization signal or a protein transduction domain. The nuclear localization signals used are classical NLS that naturally occur in proteins.

U.S. Pat. No. 6,521,456 B1 corresponding to WO 00/40742 A1 discloses a nuclear transport agent comprising a DNA-binding part that binds specifically to DNA and an extended nuclear localization signal that has a substantially neutral net charge.

WO 02/055721 A2 discloses a modular transfection system comprising a protein that is capable of forming nucleoprotein filaments (NPFs) if loaded onto a nucleic acid to be transfected. The NPF-forming protein may be modified with a nuclear localization signal in order to improve transport of the nucleic acid into the nucleus of eukaryotic cells.

### SUMMARY OF THE INVENTION

The problem underlying the present invention is to provide an alternative transport agent and method which are effective to allow an efficient transport of nucleic acids into eukaryotic cells.

The problem is solved by a transport agent that comprises a complex forming moiety that is capable of forming complexes with at least one nucleic acid molecule and condensing said nucleic acid molecule, and at least one nuclear localization moiety comprising at least one nuclear localization signal (NLS) and having an approximately neutral net charge. The combination of a moiety that binds and condenses nucleic acid molecules with a moiety that comprises an NLS but has a substantially neutral net charge leads to an effective transport agent which allows for a highly efficient transfer of molecules into eukaryotic cells. Compacting or condensing the nucleic acid to be transported facilitates the transfer through the cell membrane as well as through the nuclear pores. The compact volume, shape and size dimensions of the complex of the complex forming moiety and the nucleic acid to be transported are thus a crucial feature of the transport agent according to the invention. Additionally, the NLS sequence that serves for the transfer of said complex into the nucleus of the cell does not mediate non-specific binding to the nucleic acid since the nuclear localization moiety has an approximately neutral net charge.

So-called non-classical NLS, as for example the NLS from influenza virus nucleoprotein (Wang et al., 1997, Neumann et al., 1997), may be used in the nuclear localization moiety. Non-classical NLS do not possess a large excess of positive charges or do not reach the nucleus via the classic route of transport. However, non-classical NLS may be less efficient in mediating nuclear transport and have by far not as extensively been shown to transport cargos other than proteins. The only case in which nuclear transport of DNA with the help of a non-classical NLS is discussed is the publication by Subramanian et al. (1999) cited above. A 38 amino acid non-classical NLS (the M9 sequence of heterogeneous nuclear ribonucleoprotein) is coupled to a scrambled version of classical NLS of the Simian Virus large T-antigen. This peptide complexed with DNA leads to an enhancement of expression when transfected together with cationic liposomes as compared to the cationic liposomes plus DNA alone. This enhancement is mostly not an effect of complexing the M9 sequence to DNA since said peptide enhances the transfection efficiency only by a factor of 1.1-1.3 as compared to a mixture of DNA and peptides of both sequences added separately to DNA. The M9 sequence alone is not likely to bind DNA since it has only two positively charged aminoacids in 39 amino acids. In addition the synthesis of a 39 amino acid peptide is more complicated and expensive than e.g. a 14 amino acid peptide (NLS-2).

The nuclear localization signal as it is used in the transport agent according to the present invention is preferably a “classical” nuclear localization signal. Classical NLS sequences have a positive net charge as the positive charges of the basic amino acids in the core sequence are an essential part of most native nuclear localization signals. But it is a drawback of classical nuclear localization signals that they tend toward binding of nucleic acids via their positive charges so that these charges are masked and their function as signals for the nuclear transport machinery is significantly impaired. According to the invention, this drawback of classical NLS is eliminated by neutralizing the positive charges of the NLS so as to obtain a nuclear localization moiety that has a substantially neutral net charge. The neutral nuclear localization moiety including the classical NLS does not bind nucleic acids non-specifically and therefore transfer of the nucleic acid to be transported into the nucleus can be optimized.

The NLS of the nuclear localization moiety may be a bipartite nuclear localization signal, i.e. a signal that consists of two separate cationic amino acid sequences.

In a preferred embodiment, the nuclear localization signal comprises a core sequence that is capable of mediating transport of said nucleic acid into the nucleus of a cell, said core sequence having a positive net charge. “Core sequence” in this context means an amino acid sequence that corresponds to a classical NLS sequence and that is sufficient to mediate nuclear transport.

In an advantageous embodiment of the invention, the nuclear localization moiety further comprises at least one charge carrier that has a negative net charge. The charge carrier may be a short peptide or any other charged molecule. In a preferred embodiment, the charge carrier comprises at least one negatively charged amino acid.

Preferably, the approximately neutral net charge of the nuclear localization moiety is achieved by at least partially neutralizing the positive net charge of the core sequence with one or more charge carrier(s). The charge carriers, e.g. acidic amino acids, are introduced into the nuclear localization moiety by coupling them to the core sequence of the NLS, i.e. the charge carriers flank the core sequence. The flanking charge carrier(s) is (are) coupled directly to the core sequence or spaced in intervals of one or more residues, for example in the case of amino acids by neutral amino acids. In any case, the charge carrier(s) should be located within the nuclear localization moiety in vicinity of the positive charges of the core sequence in order to ensure efficient neutralization of these charges. The flanking charge carrier(s) can be coupled to the N-terminus and/or C-terminus of the NLS or core sequence. In the case of bipartite NLS the flanking charge carrier(s) may also be introduced between the two parts of the signal sequences.

In order to effectively avoid non-specific binding of the nuclear localization moiety to nucleic acids the nuclear localization moiety should at most have a 3-fold positive net charge as NLS sequences having more than three positive amino acids actually bind to DNA.

In a preferred embodiment, the nuclear localization signal comprises a native amino acid sequence that has a positive net charge as present in all naturally occurring classical nuclear localization signals.

With the transport agent according to the invention resting or slowly dividing primary cells can be efficiently transfected to a percentage that allows subsequent analysis.

Most cells freshly isolated from the body of an animal or human (primary cells) do not divide at all or so rarely that DNA, after it has been transported across the cellular membrane successfully, is inactivated before it reaches the nucleus and can be expressed. So far this has led to most primary cells being untransfectable unless they were artificially stimulated to proliferate in culture. It is one of the unavoidable consequences that these cells then deviate from their original state. A method for the transfection of primary cells permits the analysis of genetic material under the original conditions of a body cell. This is of paramount importance for the investigation of genetic mechanisms and the study of processes inside of a body cell. Provision of the transport agent according to the invention is also an essential step toward a completely artificial gene transfer system for gene therapy. Such a gene transfer system must possess three functional components: one component for the passage of DNA through the cellular membrane, for which cationic lipids and cationic polymers have proved to be relatively suitable. It has to contain a second component for the transfer of the DNA into the nucleus of the (usually non-dividing) target cells and a third component that mediates the integration of the DNA into the genome. According to the present invention an efficient transport agent and method is described that can serve at least as the second component and to a lesser extend also as the first. A completely artificial gene transfer vehicle that can be employed in gene therapy will in all likelihood be easier and less expensive to produce and easier to handle than the viral systems currently applied, and it is not subject to the immanent risks of these systems. Gene therapeutic approaches have been suggested, for example, for the treatment of cancer, AIDS and various hereditary diseases and will play a significant role in medicine. The transport agent according to the present invention also increases the transfection efficiency in cultured cells. It does so by making those cells accessible for the uptake of DNA that do not divide in the time slot between passage of the DNA through the cellular membrane and analysis. This is important because even for many established cell lines an increase in transfection efficiency would facilitate the analysis and help to lower costs due to the reduced amount of cell material required. Of course, this is also true for all stages in between primary cells and established cell lines.

Preferably, the complex forming moiety comprises a basic peptide, a basic polypeptide or a basic protein and/or a cationic peptide, a cationic lipid or a non-lipid cationic polymer. As non-lipid cationic polymer polyethylenimine (PEI) may be used. In an advantageous embodiment of the invention, the complex forming moiety comprises Polylysine, Polyarginine, Polyornithine or Polyhistidine and/or any polypeptide composed of any mixture of the amino acids Lysine, Arginine, Ornithine and Histidine. Polypeptides comprising both moieties are easy to produce in a one step peptide synthesis.

In an advantageous embodiment of the invention, the complex forming moiety comprises at least one amino acid sequence according to SEQ ID NO:17, SEQ ID NO:18, SEQ ID NO:19, SEQ ID NO:20, SEQ ID NO:21, SEQ ID NO:22 and/or SEQ ID NO:23.

Alternatively, the complex forming moiety may comprise a protein belonging to the High Mobility Group (HMG) protein or Histone family. HMG proteins and histones are basic proteins capable of binding and condensing DNA (WO 97/12051 A) and are thus a suitable component of the nuclear localization moiety. Human variants of these proteins may be used as potentially non-immunogenic first moieties of the transport agent if used in conjunction with gene therapy.

In an advantageous embodiment of the invention, the nuclear localization moiety comprises at least one amino acid sequence according to SEQ ID NO:2, SEQ ID NO:3, SEQ ID NO:4, SEQ ID NO:5, SEQ ID NO:8, SEQ ID NO:9, SEQ ID NO:10, SEQ ID NO:11, SEQ ID NO:12 and/or SEQ ID NO:15.

The transport agent according to the invention may additionally comprise two or more nuclear localization moieties. These additional moieties may be identical or at least have a similar structure, i.e. may merely differ in respect of one or a few residues. Using two or more nuclear localization moieties significantly improves the transfer of nucleic acids into the nucleus and therefore leads to an increased transfection efficiency.

The complex forming moiety and the at least one nuclear localization moiety are preferably linked via at least one spacer molecule. This spacer molecule may comprise 8-amino-3,6-dioxaoctanoic acid and/or NH₂-Polyethyleneglykol-27-COOH. In an advantageous embodiment of the invention the spacer molecule comprises at least one monomer, preferably a multimer, in particular a dimer, trimer or tetramer.

The present invention further concerns a composition for transfecting eukaryotic cells, said composition comprising the transport agent according to the invention and at least one cationic polymer and/or cationic lipid capable of transporting the resulting complex from outside of the cell into the cytoplasm.

The invention also relates to a pharmaceutical composition comprising the transport agent according to the invention and common adjuvants and/or carrier substances.

The problem underlying the invention is further solved by a method for producing a transport agent for transporting nucleic acids into eukaryotic cells, said method comprising the steps of

a) providing a complex forming moiety that is capable of forming complexes with at least one nucleic acid molecule and condensing said nucleic acid molecule,
b) providing a nuclear localization moiety comprising at least one nuclear localization signal having a positively charged core sequence that is capable of mediating nuclear transport,
c) neutralizing at least partially the positive net charge of said core sequence by coupling to said core sequence at least one charge carrier having a negative net charge in order to achieve an approximately neutral net charge of said nuclear localization moiety, and
d) linking said complex forming moiety to said nuclear localization moiety directly or via a spacer comprising, e.g., of further (neutral) amino acids, 2-aminoethoxy-2-ethoxy acetic acid (AEEA) or polyethylene glycol (PEG). The moieties may be linked by covalent bonds, hydrogen bonds or ionic interactions.

In an alternative embodiment of the above method, in step a) the complex forming moiety may already be provided in complex with a nucleic acid to be transfected.

The method according to the invention can be advantageously used for producing the transport agent according to the invention as described above.

The transport agent(s) according to the invention can be advantageously used in a method for transporting nucleic acids into eukaryotic cells, wherein a nucleic acid to be transported is incubated with a transport agent according to the invention in order to form a complex comprising said nucleic acid and said transport agent, and incubating said complex with at least one eukaryotic cell. Alternatively, the nucleic acid to be transported can at first be incubated with the complex forming moiety of the transport agent which is then coupled to the nuclear transport moiety before incubating the resulting complex with the cells.

The transport agent(s) according to the invention can further be advantageously used in a method for transporting nucleic acids into the nucleus of eukaryotic cells, wherein a nucleic acid to be transported is incubated with a transport agent according to the invention in order to form a complex of said nucleic acid and said transport agent and in addition with at least one cationic polymer and/or cationic lipid capable of transporting said complex from outside the cell into the cytoplasm, and incubating the resulting complex with at least one eukaryotic cell. Alternatively, the nucleic acid to be transported can at first be incubated with the complex forming moiety of the transport agent which is then coupled to the nuclear transport moiety before incubating the resulting complex with the cationic polymer and/or cationic lipid.

In a preferred embodiment of the invention, the above method(s) are accomplished with eukaryotic cells that are resting, slowly dividing or non-dividing cells, preferably primary cells and or at least one DNA molecule as nucleic acid.

Various and preferred embodiments of the invention are described below in detail.

### FIGURES

FIG. 1 shows an electrophoretic mobility shift assay in agarose gel.

1 μg DNA was incubated with peptide (μg amounts as indicated in the figure) in water in a total volume of 20 μl at 20° C. for 30 min. After that loading dye was added, each sample filled into a slot of a 0.6% agarose gel (Tris-acetate/EDTA as running buffer, pH 8.5). Electrophoresis was run at 90V for 120 min. P=1 μg plasmid DNA without peptide. M=kb marker (bands indicating 1.5, 2, 3, 4, 5, and 6 kb). The gel was stained with Ethidiumbromide for 10 min, then documented using a BioRad Gel-Doc System.

FIG. 2 shows another electrophoretic mobility shift assay in agarose gel.

1 μg DNA was incubated with 1 nM peptide in water in a total volume of 20 μl at 20° C. for 30 min. After that loading dye was added, each sample filled into a slot of a 0.6% agarose gel (Tris-acetate/EDTA as running buffer, pH 8.5). Electrophoresis was run at 90V for 120 min. P=1 μg plasmid DNA without peptide. M=kb marker (bands indicating 1.5, 2, 3, 4, 5, and 6 kb). The gel was stained with Ethidiumbromide for 10 min, then documented using a BioRad Gel-Doc System.

FIGS. 3 and 4 show band-shifts of plasmid-DNA complexed with positively charged peptides coupled to NLS, in Opti-MEM.

Plasmid DNA (90 ng per sample) was incubated with different amounts (numbers above each lane indicate pico-mole amounts per sample) of positively charged peptides (see list of peptide sequences for details) in Opti-MEM as described below.

FIG. 5 shows a bar chart of the transfection of HUVEC (Human Umbilical Vein Endothelial Cells) with ExGen500 in combination with K16U3-NLS1 and

K16U3-NLS2, 24 hours post transfection.

Cells were grown and seeded as described below. Transfection was carried out with 1.5 μg DNA per sample and the amount of peptide indicated in the figure. FACS data of duplicate samples are shown. 160 pmol peptide for 1.5 μg of DNA resembles 14 pmol for 135 ng DNA used for the gel-shift assay shown in FIGS. 4 A and B.

FIG. 6 shows a bar chart of the transfection of HUVEC with ExGen500 in combination RKKU3-NLS1 and RKKU3-NLS2, 24 hours post transfection. Cells were grown and seeded as described below. Transfection was carried out with 1.5 μg DNA per sample and the amount of peptide indicated in the figure. FACS data of duplicate samples are shown. 100 pmol peptide for 1.5 μg of DNA resembles 9 pmol for 135 ng DNA used for the gel-shift assay shown in FIGS. 4 C and D.

FIG. 7 shows a bar chart of the transfection of HUVEC with ExGen500 in combination with RKK-PEG27-NLS2, 24 hours post transfection.

Cells were grown and seeded as described below. Transfection was carried out with 1.5 μg DNA per sample and the amount of peptide indicated in the figure. Mean values of the duplicate samples are shown. 120 pmol peptide for 1.5 μg of DNA resembles 11 pmol for 135 ng DNA used for the gel-shift assay shown in FIG. 4 E.

FIG. 8 shows a bar chart of the transfection of HUVEC with HiFect in combination with RKKU3-NLS1 and RKKU3-NLS2, 24 hours post transfection.

Cells were grown and seeded as described below. Transfection was carried out with 1.5 μg DNA per sample and the amount of peptide indicated in the figure. FACS data of duplicate samples are shown. 200 pmol peptide for 1.5 μg of DNA resembles 12 pmol for 90 ng DNA used for the gel-shift assay shown in FIGS. 3 C and D.

FIG. 9 shows a bar chart of the transfection of HUVEC with HiFect in combination with RKK-PEG27-NLS2, 24 hours post transfection.

Cells were grown and seeded as described below. Transfection was carried out with 1.5 μg DNA per sample and the amount of peptide indicated in the figure. FACS data of mean values of the duplicate samples are shown. 200 pmol peptide for 1.5 μg of DNA resembles 12 pmol for 90 ng DNA used for the gel-shift assay shown in FIG. 4 E.

FIG. 10 shows a bar chart of the transfection of neuronally differentiated PC12 cells with HiFect in combination with K16U3-NLS1 and K16U3-NLS2, 72 hours post transfection.

Cells were grown and seeded as described below. Transfection was carried out with 1 μg DNA per sample and the amount of peptide indicated in the figure. FACS data of mean values of the duplicate samples are shown. 170 pmol peptide for 1 μg of DNA resembles 15 pmol for 90 ng DNA used for the gel-shift assay shown in FIGS. 3 A and B.

FIG. 11 shows a bar chart of the transfection of neuronally differentiated PC12 cells with HiFect in combination with RKK-PEG27-NLS2 and DNA-complexing peptide RKK20 without NLS.

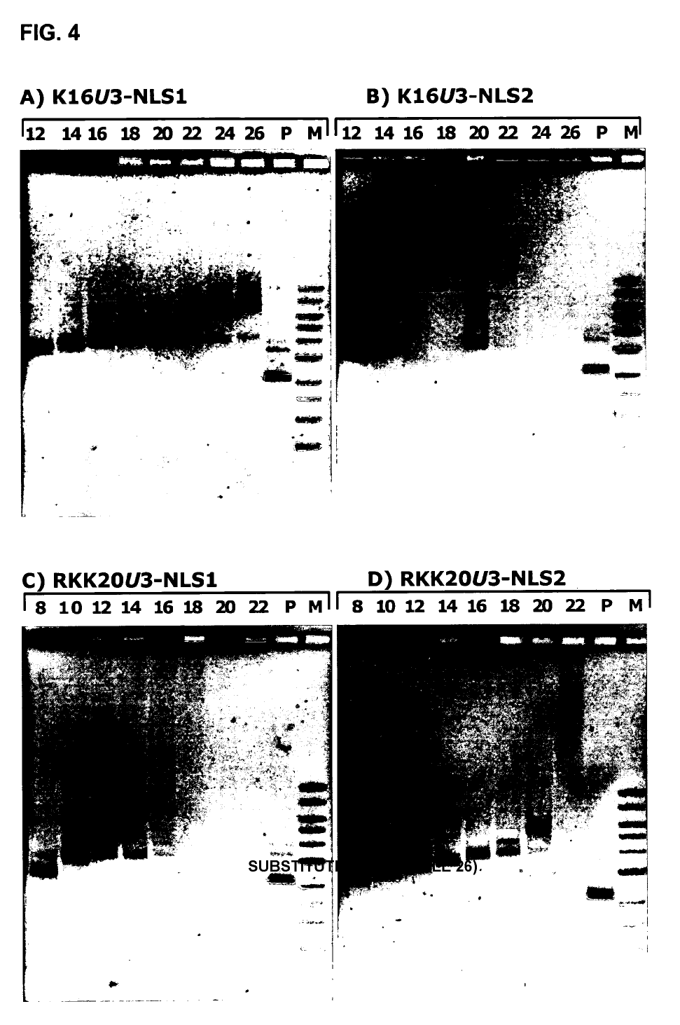

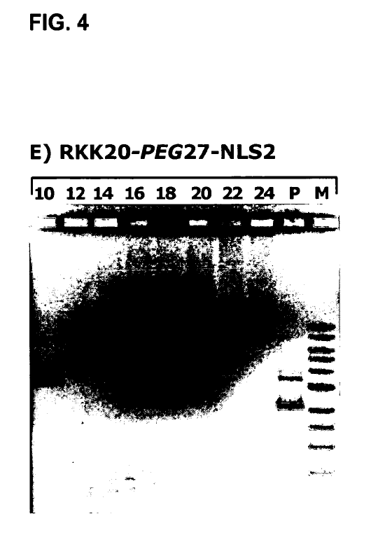

Cells were grown and seeded as described below. Transfection was carried out with 1 μg DNA per sample and the amount of peptide indicated in the figure. FACS data of mean values of the duplicate samples are shown. 260 pmol peptide for 1 μg of DNA resembles 23 pmol for 90 ng DNA used for the gel-shift assay shown in FIG. 4 E.

### EXAMPLES

### 1) Peptide Mediated Electrophoretic Mobility Shifts of DNA Upon Binding to Peptide

DNA interacting unspecifically with peptides through positively charged amino acids may mask positively charged nuclear localisation sequences (NLS) and interfere with their function in nuclear transport. In order to demonstrate that peptides with a positive net charge form higher molecular weight complexes with DNA, peptides bearing an NLS and additional negatively charged amino acids were tested for forming complexes with DNA.

Peptides were synthesized by F-moc-Chemistry and HPLC-purified to 95% purity. The carboxyl-group at the C-terminus was amidated and the N-terminal amino-group was actylated, in order to eliminate additional charges.

Sequences were the following:

NLS-1

(5-fold positively charged, SEQ ID NO: 1)

GSGSPKKKRKVGSG
SV40 large T-antigen core NLS (PKKKRKV)
with arbitrarily chosen flanking amino acids
(glycine and serine)
NLS-2

(no net charge, SEQ ID NO: 2)

EEDTPPKKKRKVED
SV40 large T-antigen core NLS (PPKKKRKV) with EED
N-terminal flanking region of the NLS form Polyoma
virus VP2-protein
NLS-3

(1-fold negatively charged, SEQ ID NO: 3)

MASQGTKRSYEQMETDGERQYC
NLS of the Influenza virus nuclear
protein
SV21

(2-fold positively charged, SEQ ID NO: 4)

GKPTADDQHSTPPKKKRKVED
Mutated part of the SV40 large
T-antigen bearing the core NLS
SV27

(no net charge, SEQ ID NO: 5)

GKPSSDDEATADSQHSTPPKKKRKVED
Natural part of the SV40 large T-antigen
bearing the core NLS

Lyophilized peptides were solved in distilled water to 1 mg/ml.

DNA was pmaxGFP plasmid DNA (4.4 kb size).

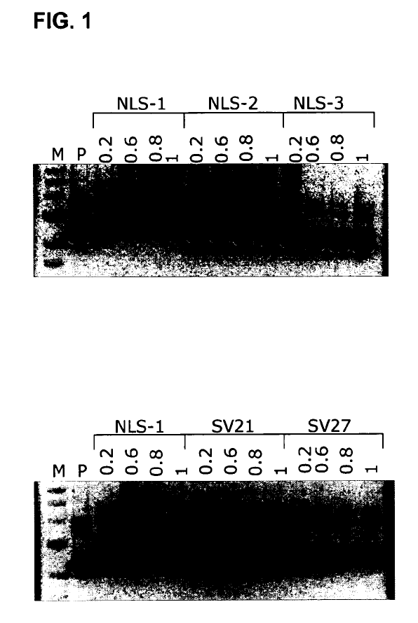

The NLS-1 peptide due to its 5-fold positive charge does bind to DNA and forms aggregates, therefore altering the electrophoretic mobility of DNA, causing a shift in apparent molecular weight (bands are running at higher molecular weight). All other peptides (of neutral charge, with one negative net charge or a 2-fold positive net charge) do not cause any alteration of the electrophoretic mobility of the DNA even at higher amounts of peptide (FIG. 1). The NLS on the NLS-1 peptide may be masked and altered in its capacity to mediate nuclear translocation, whereas all other peptides are still functioning in nuclear import.

### 2) Peptide Mediated Electrophoretic Mobility Shifts of DNA Upon Binding to Peptides with Different Positive Net Charges

DNA interacting unspecifically with peptides through positively charged amino acids may mask positively charged nuclear localisation sequences (NLS) and interfere with their function in nuclear transport. In order to demonstrate what positive net charge on a peptide is necessary to form higher molecular weight complexes with DNA, peptides bearing an NLS and additional negatively charged amino acids were tested for forming complexes with DNA. Peptides were designed with different positive net charges, 5+, 4+, 3+, 2+, 0±, by exchanging neutral amino acids for acidic ones or vice versa acidic for neutral amino acids

Peptides were synthesized by F-moc-chemistry and HPLC-purified to 95% purity. The carboxyl-group at the C-terminus was amidated and the N-terminal amino-group was actylated, in order to eliminate additional charges.

Sequences were the following:

NLS1(5+) (=NLS-1, see above)

(5-fold positively charged, SEQ ID NO: 1)

GSGSPKKKRKVGSG
SV40 large T-antigen core NLS (PKKKRKV) with
arbitrarily chosen flanking amino acids (glycine
and serine)
NLS1(4+)c

(4-fold positively charged, SEQ ID NO: 6)

ESGSPKKKRKVGSG
NLS1(4+)d

(4-fold positively charged, SEQ ID NO: 7)

GSGSPKKKRKVDSG
NLS1(3+)c

(3-fold positively charged, SEQ ID NO: 8)

ESGSPKKKRKVDSG
NLS1(3+)d

(3-fold positively charged, SEQ ID NO: 9)

EDGSPKKKRKVGSG
NLS1(3+)e

(3-fold positively charged, SEQ ID NO: 10)

GSGEPKKKRKVDSG
NLS2(0±) (=NLS-2, see above)

(no net charge, SEQ ID NO: 2)

EEDTPPKKKRKVED
SV40 large T-antigen core NLS (PPKKKRKV) with EED
N-terminal flanking region of the NLS form
Polyoma virus VP2-protein
NLS2(3+)a

(3-fold positively charged, SEQ ID NO: 11)

GSDTPPKKKRKVES
NLS2(3+)b

(3-fold positively charged, SEQ ID NO: 12)

GSGTPPKKKRKVED
NLS2(4+)a

(4-fold positively charged, SEQ ID NO: 13)

GESTPPKKKRKVGS
NLS2(4+)b

(4-fold positively charged, SEQ ID NO: 14)

GSGTPPKKKRKVES
SV21(2+) (=SV21, see above)

(2-fold positively charged, SEQ ID NO: 4)

GKPTADDQHSTPPKKKRKVED
Mutated part of the SV40 large T-antigen bearing
the core NLS
SV21(3+)e

(3-fold positively charged, SEQ ID NO: 15)

GKPTAGDQHSTPPKKKRKVED
SV21(4+)e

(4-fold positively charged, SEQ ID NO: 16)

GKPTADDQHSTPPKKKRKVSG

Lyophilized peptides were solved in distilled water to 0.1 mM.
DNA was pmaxGFP plasmid DNA (4.4 kb size).

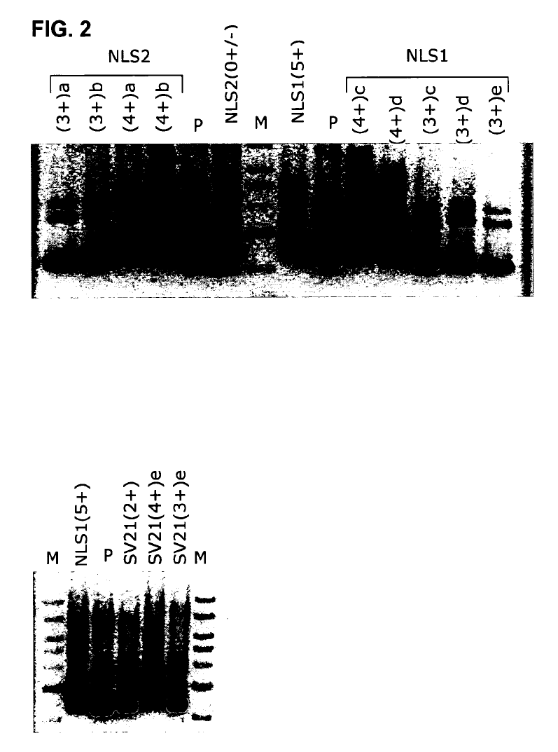

The NLS-1 peptide due to its 5-fold positive charge and the peptides with a 4-fold positive net charge do bind to DNA and form aggregates, therefore altering the electrophoretic mobility of DNA, causing a shift in apparent molecular weight (bands are running at higher molecular weight). All other peptides (of neutral charge or with a 3-fold positive net charge) do not cause any alteration of the electrophoretic mobility of the DNA even at higher amounts of peptide (FIG. 2). The NLS on the NLS-1 peptide may be masked and altered in its capacity to mediate nuclear translocation, whereas all other peptides are still functioning in nuclear import.

### 3) DNA-Band Shifts

### Binding of Positively Charged Peptides and Peptide-NLS to DNA

Several positively charged peptides, as well as positively charged peptides bearing a positively charged NLS (NLS1) or a neutral NLS (NLS2) were tested for their ability to complex DNA and form higher aggregates, visible as shifts of the electrophoretic mobility of the DNA.

Peptides were synthesized by F-moc-chemistry and HPLC-purified to 95% purity. The carboxyl-group at the C-terminus was amidated and the N-terminal amino-group was actylated, in order to eliminate additional charges. U represents the spacer molecule 8-amino-3,6-dioxaoctanoic acid (UUU (U3) represents a trimer of U):

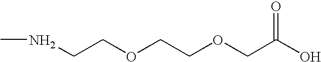

PEG27 represents the spacer NH₂-Polyethyleneglykol-27-COOH

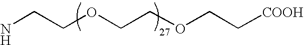

Sequences were the following:

DNA-Anchoring Peptides (complex forming
moieties):

(SEQ ID NO: 17)

| | |
|---|---|
| K16GGGS | KKKKK KKKKK KKKKK KGGGS |

(SEQ ID NO: 18 + U3)

| | |
|---|---|
| K16U3 | KKKKK KKKKK KKKKK K-UUU |

(SEQ ID NO: 19)

| | |
|---|---|
| K20GGGS | KKKKK KKKKK KKKKK KKKKK GGGS |

(SEQ ID NO: 20)

| | |
|---|---|
| K25GGGS | KKKKK KKKKK KKKKK KKKKK KKKKK GGGS |

(SEQ ID NO: 21)

| | |
|---|---|
| RKG18GGGS | RKGKKV RKGKKV RKGKKV GGGS |

(SEQ ID NO: 22)

| | |
|---|---|
| RKG24GGGS | RKGKKV RKGKKV RKGKKV RKGKKV GGGS |

(SEQ ID NO: 23)

| | |
|---|---|
| RKK20GGGS | RKKRK RKKRK RKKRK RKKRK GGGS |

Anchor Peptides with NLS-Sequence Motif:

(SEQ ID NO: 18-U3-SEQ ID NO: 1)

| | |
|---|---|
| K16U3-NLS1 | KKKKK KKKKK KKKKK K-UUU-GSGSP |
| | KKKRK VGSG |

(SEQ ID NO: 18-U3-SEQ ID NO: 2)

| | |
|---|---|
| K16U3-NLS2 | KKKKK KKKKK KKKKK K-UUU-EEDTP |
| | PKKKR KVED |

(SEQ ID NO: 23-U3-SEQ ID NO: 1)

| | |
|---|---|
| RKK20U3-NLS1 | RKKRK RKKRK RKKRK RKKRK-UUU-GSGSP |
| | KKKRK VGSG |

(SEQ ID NO: 23-U3-SEQ ID NO: 2)

| | |
|---|---|
| RKK20U3-NLS2 | RKKRK RKKRK RKKRK RKKRK-UUU-EEDTP |
| | PKKKR KVED |

(SEQ ID NO: 23-PEG27-SEQ ID NO: 2)

| | |
|---|---|
| RKK20-PEG27-NLS2 | RKKRK RKKRK RKKRK RKKRK-PEG27- |
| | EEDTP PKKKR KVED |

Lyophilized peptides, provided by supplier, were dissolved in distilled water to 0.1 mM. DNA was pmaxGFP plasmid DNA, 3.7 kb size (amaxa AG, Cologne), dissolved in water at 1 mg/ml. The plasmid encodes a green fluorescent protein (GFP).

### Electrophoretic Mobility Shift Assay in Agarose Gel:

For DNA-band shift experiments 90 ng or 135 ng DNA, resp., were incubated with various amounts of peptide (indicated in the figure) in Opti-MEM (Invitrogen) in a total volume of 60 μl or 90 μl, resp., at 20° C. for 30 min. Loading dye was added before each sample was filled into a slot of a 0.6% agarose gel (40 mM Tris-acetate/1 mM EDTA as running buffer, pH 8.0). Electrophoresis was run at 90V for 120 min.

P=90 ng plasmid DNA without peptide
M=kb marker (bands indicating 0.5, 1, 1.5, 2, 3, 4, 5, 6, 8, and 10 kb).

The gel was stained with Ethidiumbromide for 10 min, then documented using a BioRad Gel-Doc System.

With increasing amounts of peptide the DNA band is gradually shifting, indicating decreased electrophoretic mobility due to higher molecular size of the peptide-DNA complexes and due to decrease in negative charge of the DNA compensated by the positively charged peptides. As the concentration of the peptide further increases, the molecular weight of the complexes increases as well. The complexes are therefore hardly moving into the gel, the bands appear right below the loading slot. The plasmid DNA is represented by two bands in the gel, one dominant band of super-coiled slightly above 2 kb, and a weaker band between 3 and 4 kb showing the relaxed plasmid DNA.

Peptides K16-GGGS and K16-U3, differing from each other by the C-terminal spacer (4 amino acid spacer GGGS, and 8-amino-3,6-dioxaoctanoic acid spacer) form a high molecular weight complex (i.e. no migration into the gel) at the same concentrations (data not shown).

The length and the amino acid composition of the peptide region interacting with the DNA have an impact on the amount of peptide needed to form a high molecular weight complex. Peptides K25GGGS and RKK20GGGS cause a total shift to decreased electrophoretic mobility at lower peptide amounts than K16GGGS and K16U3.

Peptides RKG18GGGS and RKG24GGGS need about 2 to 3 times more peptide to completely shift the DNA, probably due to the hydrophobic valine and the neutral glycine in the DNA-binding region.

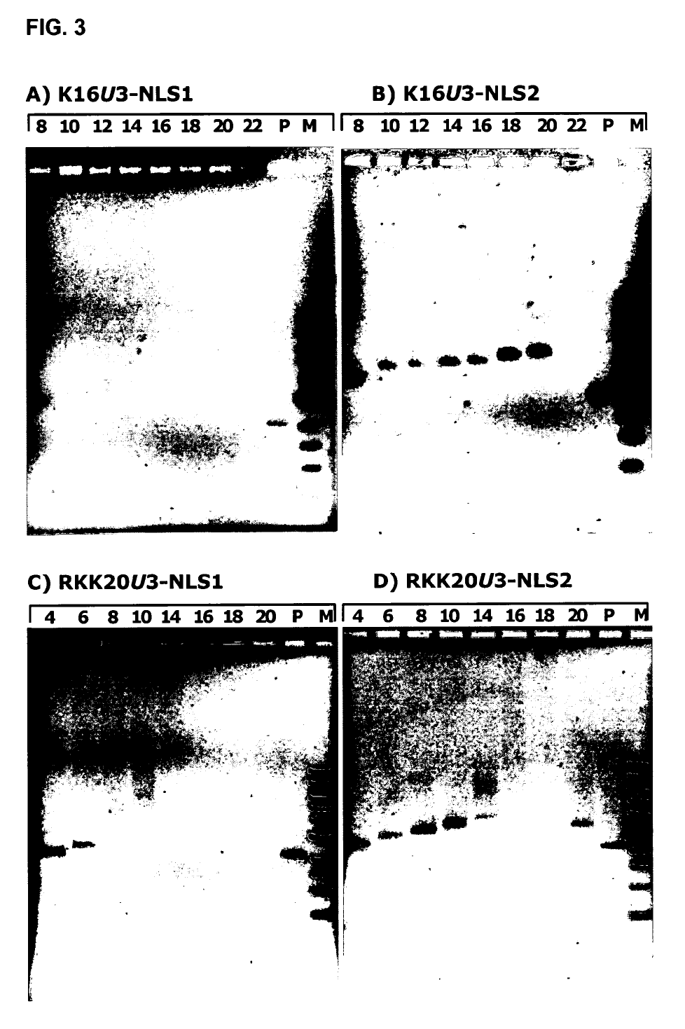

FIGS. 3 and 4 show the band shifts with the NLS-peptides in Opti-MEM or Opti-MEM/NaCl. The RKK20U3-NLS1 peptide does not behave much differently to the RKK20GGGS peptide (FIG. 4 C). In contrast adding the NLS2 to RKK20U3 seems to increase the peptide amount necessary for a total band shift (FIG. 4 D). One reason could be that the neutral NLS2, while increasing the length of the peptide, does not add any additional positive charges as NLS1 does.

The peptide RKK20-PEG27-NLS2 behaves similarly. Apparently the different spacers (8-amino-3,6-dioxaoctanoic acid versus polyethyleneglykol-27) do not have a strong influence on the DNA-binding capacity.

### 4) Transfection of Cells with DNA-Peptide Complexes

### Increase in Transfection Efficiencies Through DNA-Binding NLS-Peptides

Positively charged peptides bearing a positively charged NLS (NLS1) or a neutral NLS (NLS2) were employed for transfection of cells.

Human Umbilical Vein Endothelial Cells (HUVEC, from LONZA) were grown in EGM-2 medium (Bullet Kit, LONZA) and seeded in 12-well plates one day before transfection at a density of 1.2×10⁵ cells per well in 1 ml EGM-2 medium (LONZA).

PC12 cells (from ATCC) were grown in F-12K medium (ATCC) with 15% horse serum (Gibco) and 2.5% foetal calf serum (ATCC). Cells were seeded in 12-well plates; 5×10⁴ coated with collagen (from rat tail (SIGMA), 60 μg per well, dissolved in 0.005% acetic acid/30% ethanol at 60 μg per ml). After one day of culture nerve growth factor (NGF-β from SIGMA, 100 ng/ml final concentration) was added to differentiate neuronal cells. 7 days after start of differentiation, cells were used for transfection.

Transfection reagents were ExGen500 (Fermentas) and HiFect (amaxa) used according to manufacturer's protocols. Plasmid-DNA (1.5 μg), coding for green fluorescent protein (pmaxGFP, amaxa) was complexed with peptides (amounts as indicated in the figures) for 30 min at room temperature prior to mixing with transfection reagent.

For transfection with HiFect complex formation of DNA and peptide was done in 250 μl Opti-MEM (Invitrogen) for each sample.

For transfection with ExGen500 complex formation was done in 30 μl 150 mM NaCl solution. These preformed DNA-peptide complexes were added to transfection reagent (diluted in Opti-MEM or 150 mM NaCl according to manufacturer's protocols) and incubated.

Complete transfection samples were added to HUVEC in Opti-MEM, incubated for 1 h at 37° C./5% CO₂ in a humidified incubator. Medium was aspirated, cells washed with PBS twice. Fresh medium was added, and cells were incubated for another 23 h, then prepared for flow cytometry. To differentiated PC12 transfection samples were added in Opti-MEM, incubated for 1 h. After supernatant was removed fresh medium was added. Expression of maxGFP was measured by flow cytometry and expressed as transfection efficiency (percentage of GFP-positive cells of all viable cells) and X-mean (mean fluorescent intensity of the cells) 24 h after transfection (HUVEC), 48 h or 72 h after transfection in the case of PC12.

a) Transfection of Human Umbilical Vein Endothelial Cells (HUVEC) with ExGen500 and Peptide-NLS

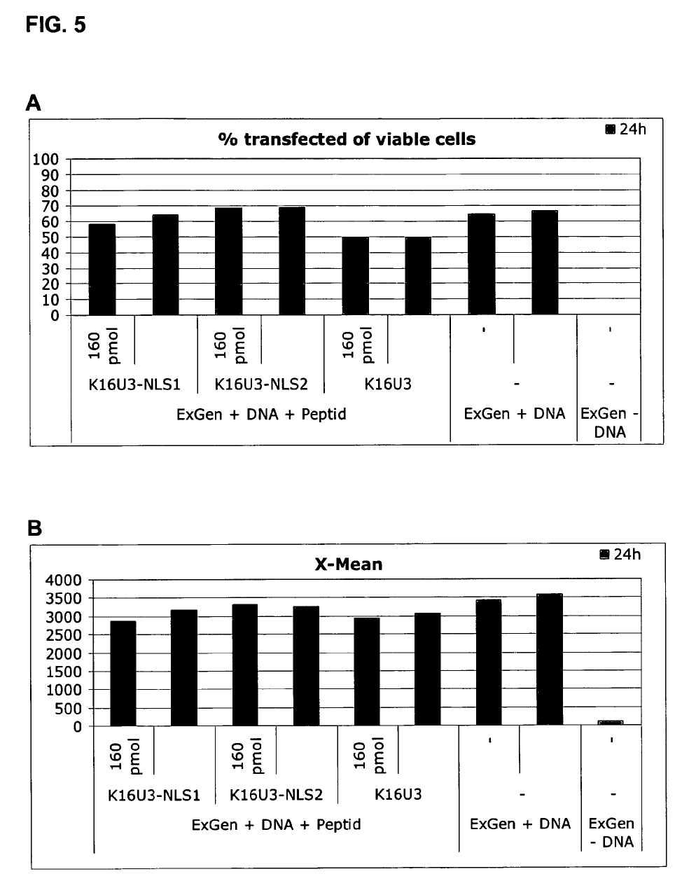

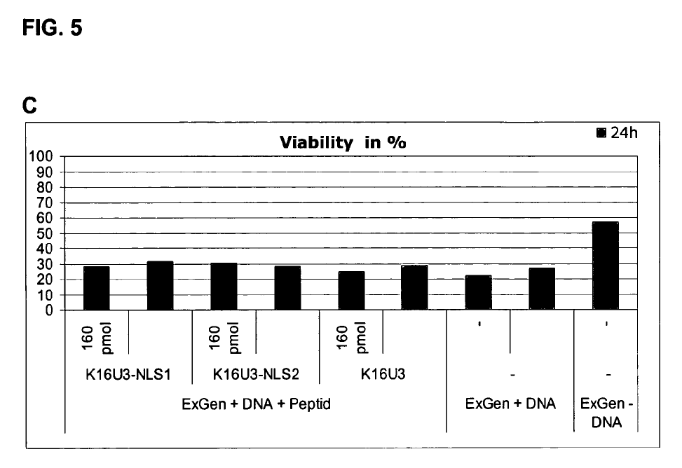

On HUVEC, transfection efficiency is increased by the oligo-lysine peptide K16U3 bearing the neutral NLS2 (69%) by a factor of 1.4 compared to peptide K16U3 without NLS (49.5%). For K16U3 bearing the positively charged NLS1 the increase of transfection efficiency is less distinctive. In comparison to ExGen without peptide, K16U3 without NLS is decreasing transfection efficiency, probably interfering with DNA transfer or expression (FIG. 5).

The amount of peptide that is most effective in increasing transfection efficiency is much lower than the peptide amount causing a complete shift of the DNA-band in the gel shift assay (FIG. 4). This is true for all the peptides tested here.

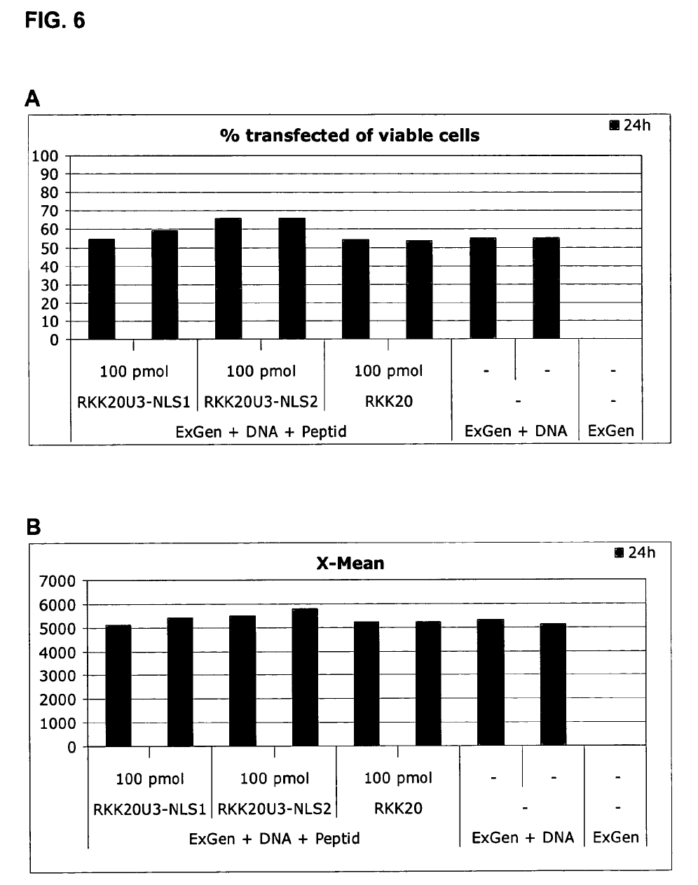

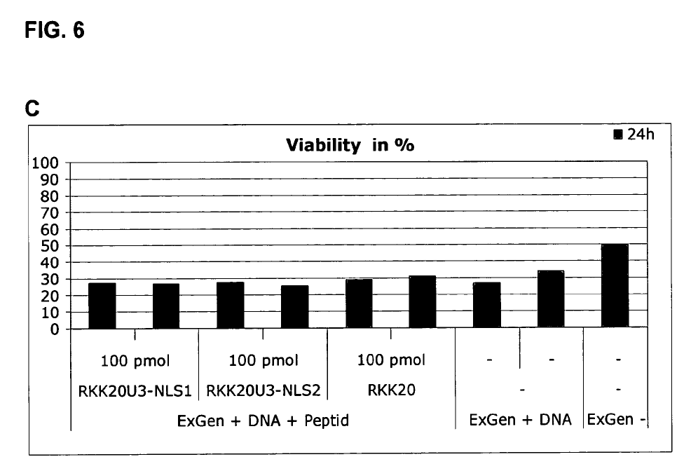

For RKKU3, the NLS2 variant yields an increase in transfection efficiency by a factor of 1.2 compared to DNA transfected without any peptide or complexed with RKK20GGGS peptide without NLS. Again, the increase is higher for the NLS2-peptide than for the NLS1-bearing peptide (FIG. 6).

The fact that a lower amount of RKKU3-NLS2 than of K16U3-NLS2 is sufficient for increase in transfection efficiency (FIG. 5, FIG. 6) may reflect differences in the DNA-complexing part of the peptides. The RKKRK-repeat of RKK20U3-NLS2 may be more effective in binding to DNA than the oligolysine repeat of K16U3-NLS2.

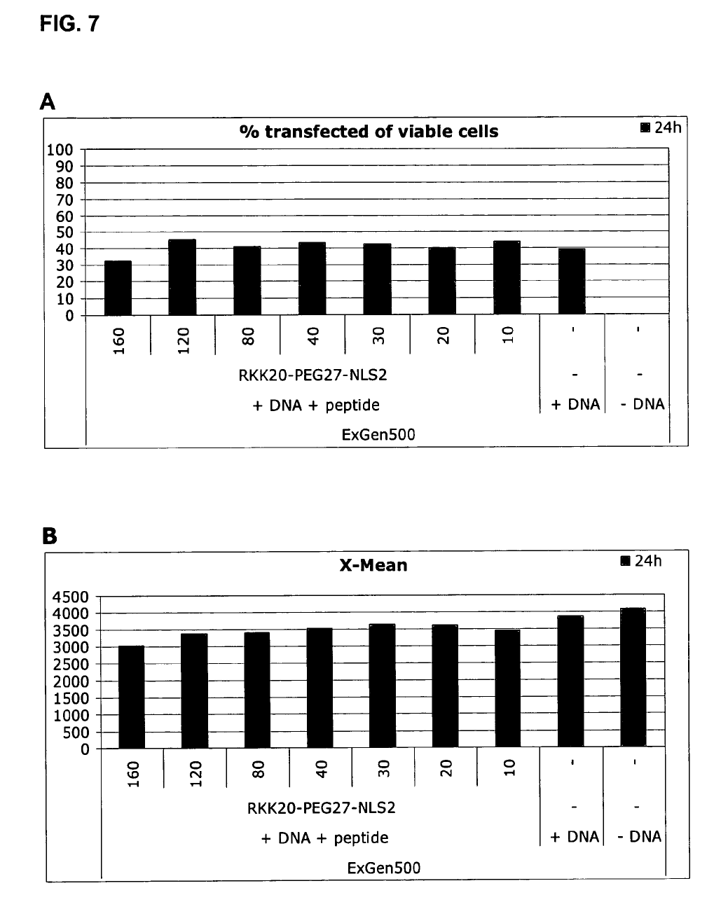

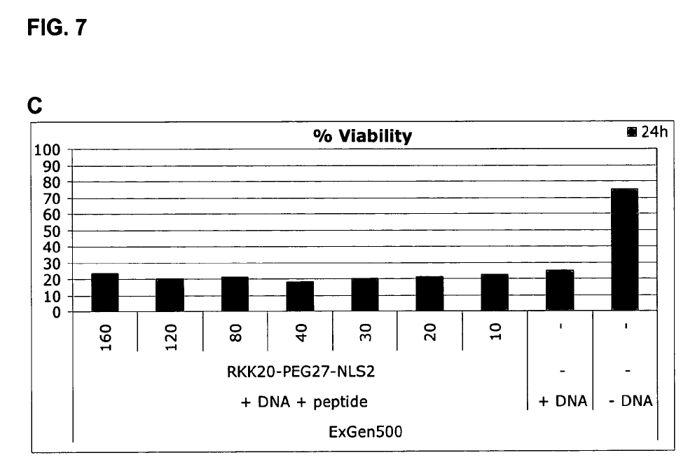

The RKK20-PEG27-NLS2 peptide with an alternative spacer yields an increase in transfection efficiency by a factor of 1.2 (FIG. 7), which is comparable to the effect of the RKKU3-NLS2 peptide (FIG. 6).

b) Transfection of Human Umbilical Vein Endothelial Cells (HUVEC) with HiFect and peptide-NLS

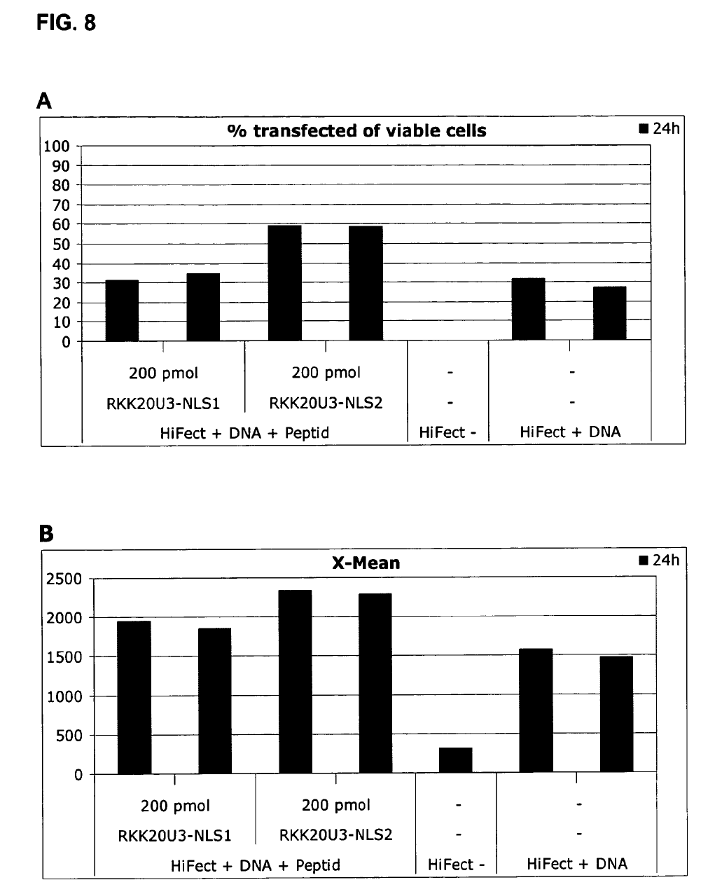

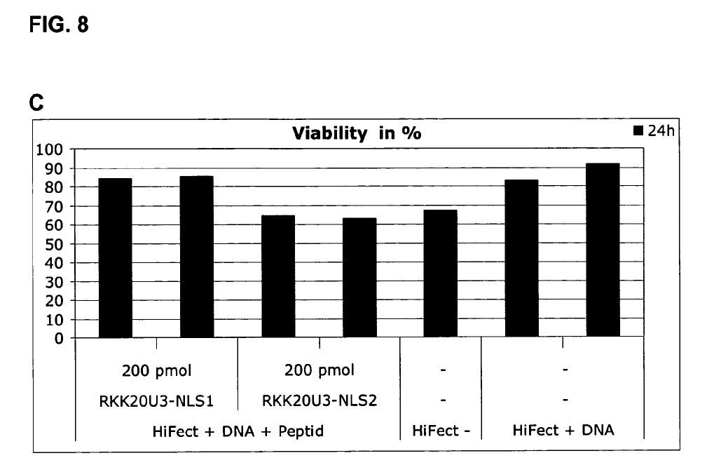

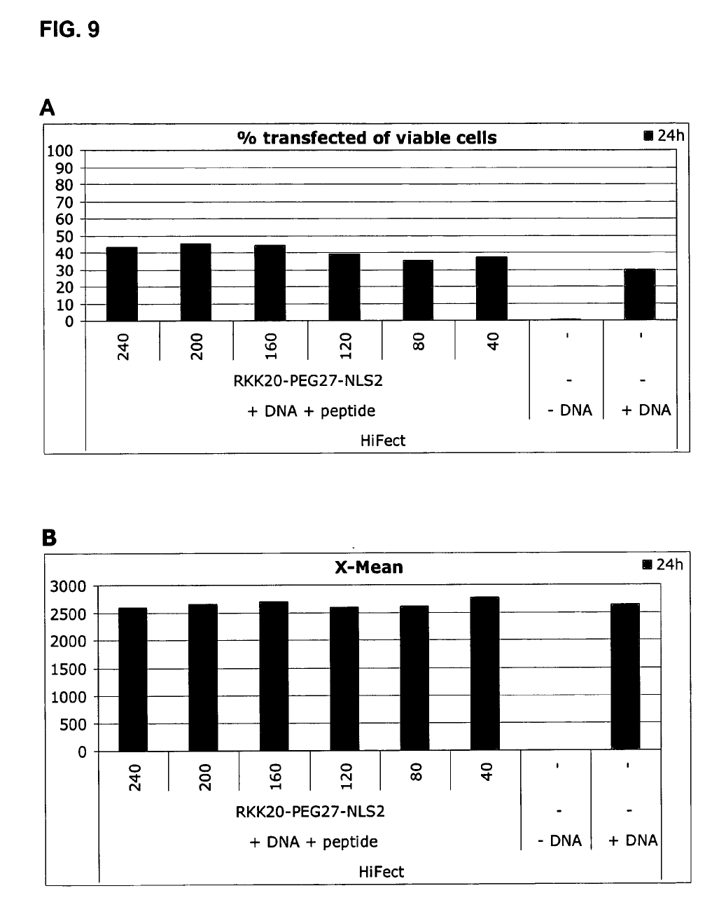

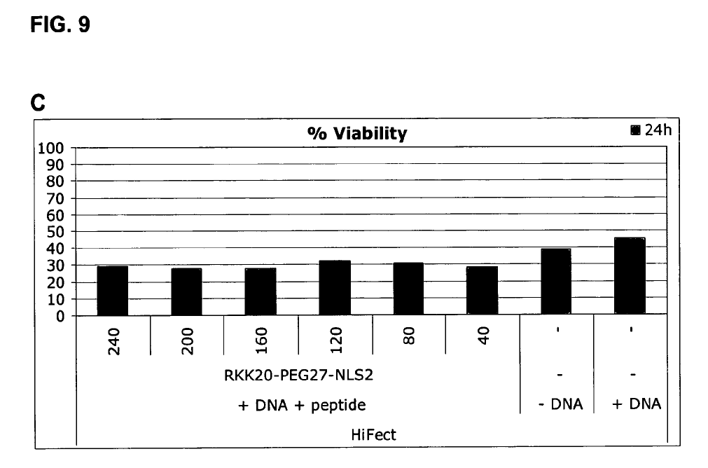

In combination with the HiFect transfection reagent, the RKK20U3-NLS2 and RKK20-PEG27-NLS2 peptides both yield an increase in transfection efficiency. RKK20U3-NLS2 yields an increase by a factor of 2 (FIG. 8), RKK20-PEG27-NLS2 an increase by a factor of 1.5 (FIG. 9).

In combination with HiFect, higher peptide amounts than for ExGen500 are necessary to reach maximum increase in transfection efficiency.

c) Transfection of PC12 Cells with HiFect and Peptide-NLS

The effect of NLS-peptides on neuronally differentiated PC12 cells was tested. This cell type is a cell-line model for neuronal cells, and represents a difficult to transfect cell type due to its resting cell status.

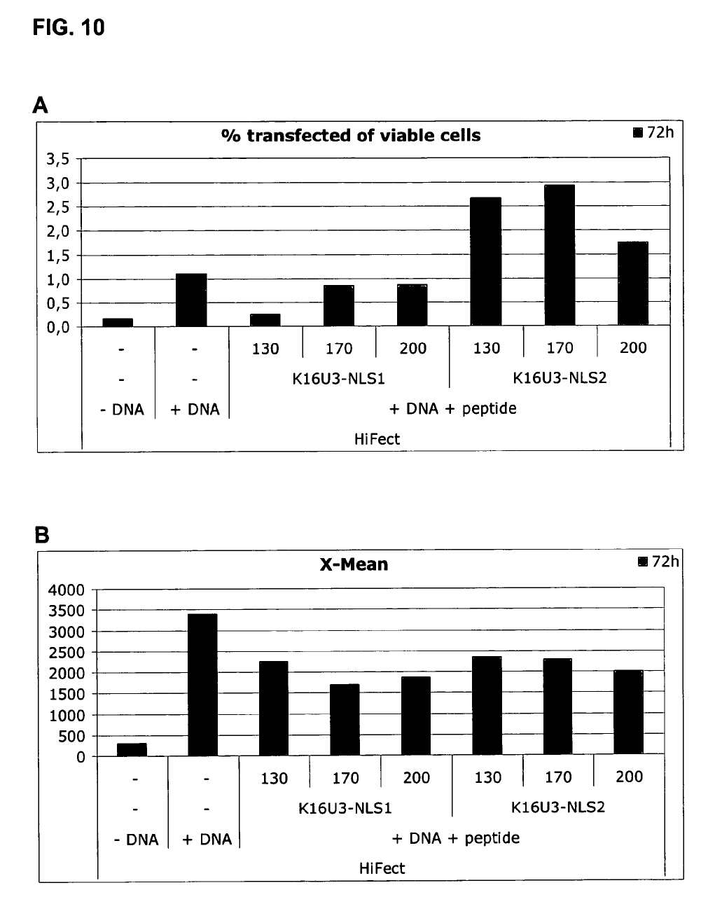

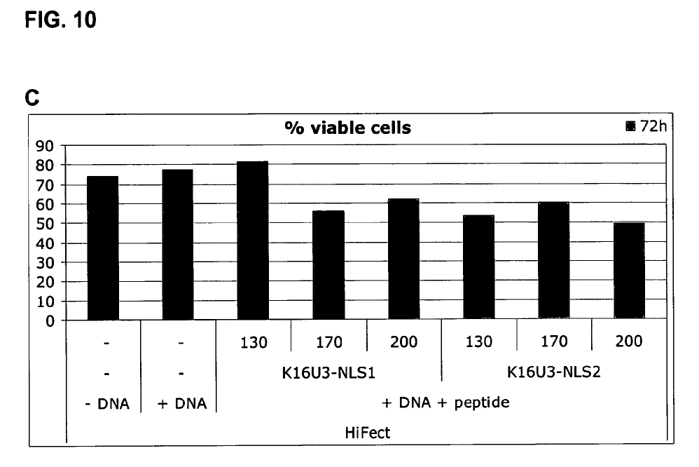

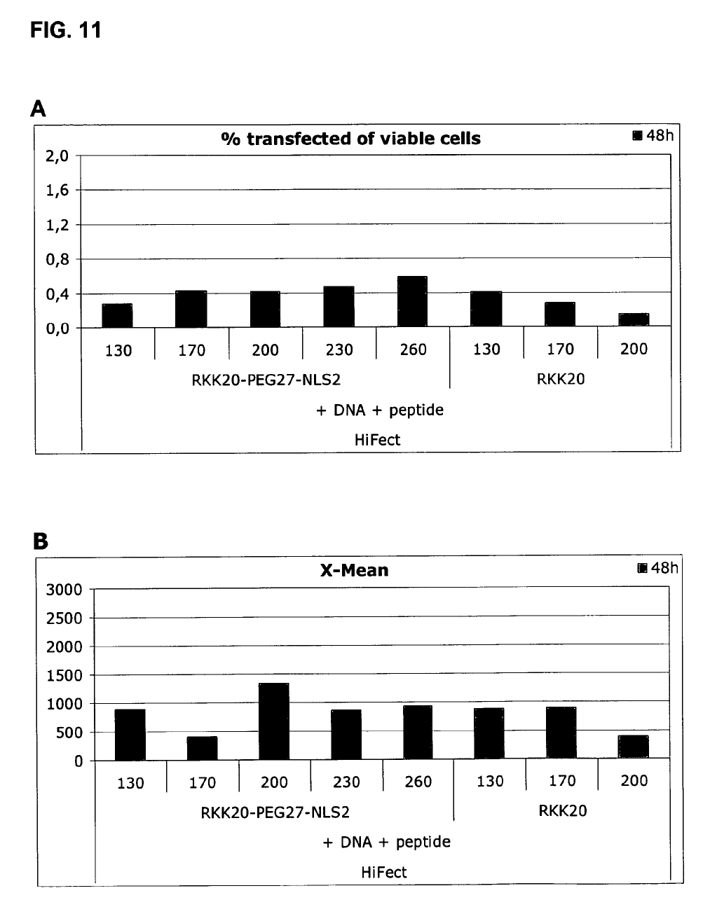

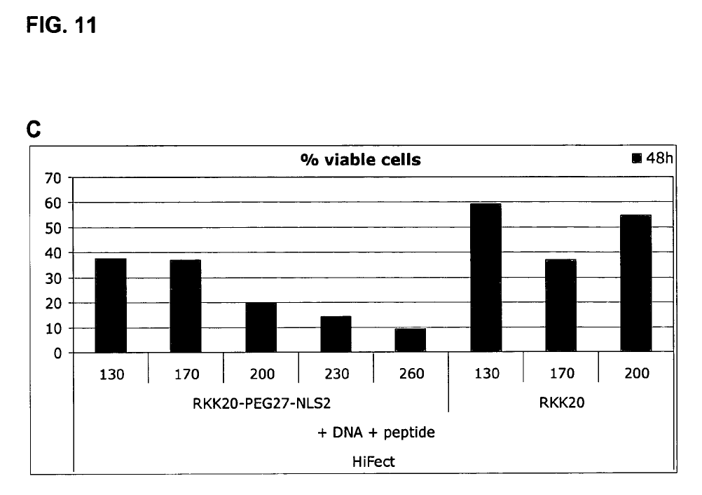

In combination with HiFect, the NLS-peptides K16U3-NLS2 and RKK20-PEG27-NLS2 both yield an increase in transfection efficiency. K16U3-NLS2 yields a maximum increase by a factor of 2.6 compared to HiFect without peptide (FIG. 10), RKK20-PEG27-NLS2 a maximum increase by a factor of 2.8 compared to peptide RKK20 without NLS (200 pmol resp., FIG. 11).

The increase in transfection efficiency by peptide-NLS2 on resting PC12 is greater than on HUVEC, demonstrating the enhanced nuclear transport properties of the inventive transport agent.

### LITERATURE

Boulikas, T. (1993). Nuclear localization signals (NLS). Crit. Rev Eukaryot Gene Expr 3, 193-227.
Emi, N. et al. (1997). Gene transfer mediated by polyarginine requires a formation of big carrier-complex of DNA aggregate. Biochem Biophys Res Comm 231, 421-424.
Görlich, D. (1998). Transport into and out of the cell nucleus. EMBO J. 17, 2721.
Nagasaki, T. et al. (2003). Can nuclear localization signals enhance nuclear localization of plasmid DNA. Bioconjug Chem 14, 282-286.
Neumann, G. et al. (1997). Nuclear import and export of influenza virus nucleoprotein. J Virol 71, 9690-9700.
Subramanian, A. et al. (1999). Nuclear targeting peptide scaffolds for lipofection of nondividing mammalian cells. Nature Biotechnology 17, 873-877.
Wang, P. et al. (1997). The NPI-1/NPI-3 binding site on the influenza a virus nucleoprotein NP is a nonconventional nuclear localization signal. J Virol 71, 1850-1856.
Yoneda, Y. et al. (1987). Synthetic peptides containing a region of SV 40 large T-antigen involved in nuclear localization direct the transport of proteins into the nucleus. Exp cell Res 170, 439-453.
Yoneda, Y. et al. (1992). A long synthetic peptide containing a nuclear localization signal and its flanking sequences of SV 40 T-antigen directs the transport of IgM into the nucleus effectively. Exp cell Res 201, 313.

1. A transport agent for transporting nucleic acids into eukaryotic cells, said transport agent comprising a complex forming moiety that forms complexes with at least one nucleic acid molecule and condenses said nucleic acid molecule, and at least one nuclear localization moiety comprising at least one nuclear localization signal and having an approximately neutral net charge.

2. The transport agent according to claim 1, wherein said complex forming moiety comprises a basic peptide, a basic polypeptide or a basic protein.

3. The transport agent according to claim 1 or 2, wherein said complex forming moiety comprises a cationic peptide, a cationic lipid or a non-lipid cationic polymer.

4. The transport agent according to claim 1, wherein said complex forming moiety comprises Polylysine, Polyarginine, Polyornithine or Polyhistidine and/or any polypeptide composed of any mixture of the amino acids Lysine, Arginine, Ornithine and Histidine.

5. The transport agent according to claim 1, wherein said complex forming moiety comprises at least one amino acid sequence according to SEQ ID NO:17, SEQ ID NO:18, SEQ ID NO:19, SEQ ID NO:20, SEQ ID NO:21, SEQ ID NO:22 and/or SEQ ID NO:23.

6. The transport agent according to claim 1, wherein said nuclear localization signal comprises a core sequence that mediates transport of said nucleic acid into a nucleus of a cell, said core sequence having a positive net charge.

7. The transport agent according to claim 1, wherein said nuclear localization moiety further comprises at least one charge carrier that has a negative net charge.

8. The transport agent according to claim 7, wherein said charge carrier comprises at least one negatively charged amino acid.

9. The transport agent according to claim 6, wherein the approximately neutral net charge of said nuclear localization moiety is achieved by at least partially neutralizing the positive net charge of the core sequence with said at least one charge carrier.

10. The transport agent according to claim 1, wherein said nuclear localization moiety has at most a 3-fold positive net charge.

11. The transport agent according to claim 1, wherein said nuclear localization signal comprises a native amino acid sequence that has a positive net charge.

12. The transport agent according to claim 1, wherein said nuclear localization moiety comprises at least one bipartite nuclear localization signal.

13. The transport agent according to claim 1, wherein said nuclear localization moiety comprises at least one amino acid sequence according to SEQ ID NO:2, SEQ ID NO:3, SEQ ID NO:4, SEQ ID NO:5, SEQ ID NO:8, SEQ ID NO:9, SEQ ID NO:10, SEQ ID NO:11, SEQ ID NO:12 and/or SEQ ID NO:15.

14. The transport agent according to claim 1, comprising two or more nuclear localization moieties.

15. The transport agent according to claim 1, wherein the complex forming moiety and the at least one nuclear localization moiety are linked via at least one spacer molecule.

16. The transport agent according to claim 15, wherein the spacer molecule comprises 8-amino-3,6-dioxaoctanoic acid and/or NH₂-Polyethyleneglykol-27-COOH.

17. The transport agent according to claim 15 or 16, wherein the spacer molecule comprises at least one monomer.

18. A composition for transfecting eukaryotic cells, said composition comprising the transport agent according to claim 1 and at least one cationic polymer and/or cationic lipid for transporting the resulting complex from outside of the cell into its cytoplasm.

19. A pharmaceutical composition comprising the transport agent according to claim 1 and common adjuvants and/or carrier substances.

20. A method for producing a transport agent for transporting nucleic acids into eukaryotic cells, said method comprising a) providing a complex forming moiety that is capable of forming complexes with at least one nucleic acid molecule and condensing said nucleic acid molecule, b) providing a nuclear localization moiety comprising at least one nuclear localization signal having a positively charged core sequence that is capable of mediating nuclear transport, c) neutralizing at least partially the positive net charge of said core sequence by coupling to said core sequence at least one charge carrier having a negative net charge in order to achieve an approximately neutral net charge of said nuclear localization moiety, and d) linking said complex forming moiety to said nuclear localization moiety directly or via a spacer.

21. The method according to claim 20 for producing the transport agent for transporting nucleic acids into eukaryotic cells, said transport agent comprising a complex forming moiety that forms complexes with at least one nucleic acid molecule and condenses said nucleic acid molecule, and at least one nuclear localization moiety comprising at least one nuclear localization signal and having an approximately neutral net charge.

22. A method for transporting nucleic acids into eukaryotic cells, wherein a nucleic acid to be transported is incubated with a transport agent according to claim 1 to form a complex comprising said nucleic acid and said transport agent, and incubating said complex with at least one eukaryotic cell.

23. A method for transporting nucleic acids into the nucleus of eukaryotic cells, wherein a nucleic acid to be transported is incubated with a transport agent according to claim 1 to form a complex of said nucleic acid and said transport agent and in addition with at least one cationic polymer and/or cationic lipid of for transporting said complex from outside the cell into the cytoplasm, and incubating the resulting complex with at least one eukaryotic cell.

24. The method according to claim 22 or 23, wherein said eukaryotic cells are resting, slowly dividing cells or non-dividing cells, preferably primary cells.

25. The method according to claim 22 or 23, wherein said nucleic acid is at least one DNA molecule.

26. The transport agent according to claim 17, wherein the spacer molecule comprises a multimer.

27. The transport agent according to claim 26, wherein said multimer is a dimer trimer or tetramer.
